(19) United States
(12) Patent Application Publication
(10) Pub. No.: US 2023/0406793 A1
(43) Pub. Date: Dec. 21, 2023

(54) FACILITY AND METHOD FOR PRODUCING A GLOBALLY USABLE ENERGY CARRIER

(71) Applicant: OBRIST TECHNOLOGIES GMBH, Lustenau (AT)

(72) Inventor: Frank Obrist, Bregenz (AT)

(21) Appl. No.: 18/035,726

(22) PCT Filed: Nov. 5, 2021
(86) PCT No.: PCT/EP2021/080701

§ 371 Date: May 5, 2023

(30) Foreign Application Priority Data
Nov. 7, 2020 (DE) 10 2020 129 374.7

(51) Int. Cl. C07C 29/151; C02F 1/26; B01D 53/14; C25B 1/04; C25B 15/08

(52) U.S. Cl. CPC C07C 29/1516; C02F 1/265; B01D 53/1475; C25B 1/04; C25B 15/081; C02F 2103/08

(57) ABSTRACT

The disclosure relates to a plant for the production of a globally usable energy carrier having a photovoltaic unit for converting solar energy into electricity, a water supply unit for the production of desalinated sea water, an electrolysis unit for the production of hydrogen connected by pipeline to the water supply unit for the supply of desalinated water, a carbon dioxide absorption unit for absorbing carbon dioxide from the ambient air, a methanol synthesis unit (34) for producing methanol connected by a pipeline to the electrolysis unit for supplying hydrogen and by a pipeline to the carbon dioxide absorption unit for supplying carbon dioxide, wherein the water supply unit unit, the electrolysis unit, the carbon dioxide absorption unit and the methanol synthesis unit each are connected to the photovoltaic unit for the supply of power and are arranged in a contiguous plant area.

The invention relates to a plant, a plant complex and a method for the production of a globally usable energy carrier. The invention further relates to a globally usable energy carrier, the use of methanol, and a system and method for the formation of a global carbon dioxide cycle.

Mobility is one of the most important prerequisites for economic success, employment and prosperity. At the same time, however, mobility also means a heavy burden on the environment from transport systems and from the global traffic volume, which has risen continuously in recent decades. It is true that the efficiency of combustion engines has improved significantly and they have become cleaner and quieter. However, the increased traffic volume continues to generate and emit large quantities of climate gases and air pollutants into the atmosphere. With the increase in traffic volume, the energy consumption of transport in Germany, for example, has more than tripled since 1960. Currently, transportation is responsible for about one-fifth of greenhouse gas emissions in Germany. What applies to environmental and climate pollution in Germany also applies to the global climate situation, which suffers not only from the burning of fossil fuels in road traffic, but also from combustion in power plants for electricity generation.

To counteract this negative development and limit climate damage, the Climate Protection Plan 2050 adopted in Germany, for example, aims to reduce annual greenhouse gas emissions in the transport sector from the current level of around 160 million metric tons of CO₂ equivalents to between 95 and 98 million metric 30 tons of CO₂ equivalents in 2030. The European Commission is also pursuing the goal of decarbonizing the European mobility system by 2050, i.e., making it greenhouse gas neutral. Success depends on whether the measures taken to achieve these goals are supported by broad sections of society and whether these measures are cost-effective. The central objective is to ensure that the mobility needs of broad sections of the population today are met in the future in a manner that is as environmentally compatible as possible. This means that a successful mobility solution must not only be technically feasible and target-oriented, but must also be measured against the costs of current mobility solutions.

In addition to the well-known fuel cell technology and electrification of vehicle drives, the use of synthetically produced fuels is an important component in the development of new mobility concepts. Methanol plays an important role as a fuel in this context. Methanol is produced by synthesizing hydrogen and carbon dioxide, which are obtained from renewable sources or in a greenhouse gas-neutral manner. For this purpose, for example, WO 2018/112654 A1 describes a process in which hydrogen is produced by electrolysis and carbon dioxide is obtained by direct separation from ambient air, which are used to produce methanol.

However, the known process is not suitable for providing an energy carrier in sufficient quantities and with the necessary economic efficiency to reduce climate pollution to a noticeable extent and at the same time meet current mobility needs.

The invention is based on the task of specifying a plant for the production of a globally usable energy carrier which creates the prerequisite for a new, global mobility concept which does not restrict current mobility needs or only slightly and is at the same time climate-neutral, wherein the new, global mobility concept should require at most the same costs as current mobility concepts which are essentially based on the combustion of fossil fuels. In particular, the plant is intended to lay the foundation for covering at least part, and in particular all, of the global energy demand by a regeneratively produced, synthetic fuel in an economical manner. The invention is further based on the task to disclose a plant complex and a method for the production of a globally usable energy carrier, a globally usable energy carrier and the use of methanol. The invention is also based on the task to disclose a system for the formation of a global carbon dioxide cycle with regeneratively produced methanol as energy carrier as well as a corresponding method.

According to the invention, this task is solved with regard to the plant by the subject matter of claim 1, with regard to the plant complex by the subject matter of claim 13, with regard to the method for producing a globally usable energy carrier by the subject matter of claim 15, with regard to the globally usable energy carrier by the subject matter of claim 16, and with regard to the use of methanol by the subject matter of claim 17. With respect to the system for forming a global carbon dioxide cycle, the task is solved by the subject matter of claim 18, and with respect to the method for forming a global carbon dioxide cycle, by the subject matter of claim 20.

Specifically, the task is solved by a plant for the production of a globally usable energy carrier with a photovoltaic unit for converting solar energy into electricity, which has a capacity, in particular peak capacity of at least 1.0 gigawatt, in particular at least 1.3 gigawatt, in particular at least 1.5 gigawatt. The plant comprises a water supply unit, in particular a seawater desalination unit for the production of desalinated water, which has an intake capacity of at least 900000 tons of seawater per year, and an electrolysis unit for the production of hydrogen, which is connected by at least one pipeline to the water supply unit, in particular seawater desalination unit for supplying water, in particular desalinated water. The plant comprises a carbon dioxide absorption unit for absorbing carbon dioxide from ambient air having an extraction capacity of at least 400000 tons of carbon dioxide per year, in particular of at least 600000 tons of carbon dioxide per year from ambient air, and a methanol synthesis unit for producing methanol, which is connected by at least one pipeline to the electrolysis unit for supplying hydrogen and by at least one pipeline to the carbon dioxide absorption unit for supplying carbon dioxide. The water supply unit, in particular seawater desalination unit, the electrolysis unit, the carbon dioxide absorption unit and the methanol synthesis unit are each connected to the photovoltaic unit for the supply of power and are arranged in a contiguous plant area with the photovoltaic unit. The photovoltaic unit is adapted to absorb at least 1500 kWh/m²a, in particular at least 2000 kWh/m²a, in particular at least 2300 kWh/m²a, in particular at least 2500 kWh/m²a, in particular at least 2700 kWh/m²a of solar energy. The methanol synthesis unit has a output capacity of at least 300000 tons, in particular of at least 450000 tons of regeneratively produced methanol per year. A output capacity of 450000 tons of regeneratively produced methanol per year is particularly preferred.

The plant according to the invention has several advantages that together predestine the plant according to the invention to become the basis for a future global energy supply that is climate neutral and economically competitive.

The invention has the advantage that the plant components provided within the scope of the invention enable location-optimization, which is important for the economic operation of the plant. Thus, the plant according to the invention comprises a water supply unit, in particular a seawater desalination unit, an electrolysis unit, a carbon dioxide absorption unit, a methanol synthesis unit and a photovoltaic unit.

According to the invention, the process-relevant units are each connected to the photovoltaic unit for power supply and are arranged together with the photovoltaic unit in a coherent plant area. This means that the individual units are arranged in spatial proximity to one another and are combined in a uniform plant. It is not necessary for the plant area to be closed. The individual units can, for example, be separated from each other by supply roads that run through the plant. This ensures that the transport of material flows and the power supply between the units are carried out with the lowest possible losses

It also means that the plant as a whole is designed in such a way that it can be positioned in a location-optimized manner and operated in a self-sufficient manner. The particular advantage of combining the seawater desalination unit and the photovoltaic unit is that the plant can be located in geographical regions, such as the Middle East or Africa, which have both high solar irradiation and access to seawater, so that on the one hand the energy supply of the plant by the photovoltaic unit and on the other hand the provision of sufficiently large quantities of water for the electrolysis unit is made possible in an economical manner.

By supplying power to the electrolysis unit exclusively through the photovoltaic unit, the plant produces hydrogen in a regenerative manner. The carbon dioxide required for methanol production is extracted from the ambient air of the plant by the carbon dioxide absorption unit. By combining the two units in the plant according to the invention, methanol production is carried out in a regenerative manner without generating carbon dioxide. Rather, even by removing the carbon dioxide from the ambient air, the carbon dioxide concentration in the atmosphere is reduced. The plant according to the invention is thus suitable to be a part of a system forming a global carbon dioxide cycle with regeneratively produced methanol as energy carrier. This means that the carbon dioxide extracted from the ambient air does not have to be disposed of or dumped, as is usually the case, for example, in the well-known storage of carbon dioxide in deep rock strata, wherein this is not excluded, for example, as an additional measure. The carbon dioxide absorbed from the ambient air is a valuable substance, which is used in the plant according to the invention for the production of a synthetic fuel, namely methanol, and can thus be fed into a carbon dioxide cycle. The plant according to the invention forms a building block for the system described and claimed in this application, which enables this carbon dioxide cycle.

The economic efficiency of the measures taken, which is necessary for the implementation of the climate targets mentioned further above, is made possible by the plant according to the invention. It is true that, compared to the production and use of hydrogen for fuel cells, the conversion losses increase due to the additional process steps required for the synthesis of methanol. However, this is offset by much greater economic benefits in terms of global infrastructure costs associated with methanol combustion compared to pure electric propulsion or fuel cell technology. The costs for expensive charging stations or for the technically complex storage of hydrogen do not apply to the combustion of regeneratively produced methanol. Storage and transport of methanol require no special measures and are comparable to the handling of conventional fuels. Another advantage of regeneratively produced methanol compared to the energy sources hydrogen or batteries is that the energy density of methanol with 4.35 kWh/liter is much higher than that of high pressure hydrogen (800 bar) with 1.25 kWh/liter, liquid hydrogen with 2.36 kWh/liter and batteries with 0.5 kWh/liter.

A comparison of current energy prices, which is intended to give an approximate indication of the energy costs of the plant according to the invention, shows that the photovoltaic unit provided as part of the plant according to the invention is an important component not only for producing synthetic fuel in a regenerative manner, but also for producing it so economically that the fuel can prevail in the competitive battle with other energy sources. The energy prices for wind energy (2.39 EURct/kWh) and hydropower energy (1.71 EURct/kWh) are currently (2020) already far below the energy prices for fossil fuels and, of course, nuclear energy. However, the energy price for electricity produced by photovoltaics is even lower, at 1.14 EURct/kWh for electricity produced in regions with high and long solar irradiation, such as the Middle East or Africa. Photovoltaic plants with an installed capacity of 2 gigawatts, for example, already exist there and are capable of producing electricity at the aforementioned price. The plant according to the invention is designed to be installed in regions where large areas cannot be used for agriculture because these are deserts or steppes, so that sufficiently large areas are available for a correspondingly large photovoltaic unit. According to the invention, therefore, the capacity, in particular the peak capacity of the photovoltaic unit is at least 1.0 gigawatt, in particular at least 1.3 gigawatt, in particular at least 1.5 gigawatt. The seawater desalination unit for producing desalinated water is designed for an intake capacity of at least 900000 tons of seawater per year. The carbon dioxide absorption unit is designed according to the invention for an extraction capacity of at least 400000 tons of carbon dioxide per year, in particular of at least 600000 tons of carbon dioxide per year from ambient air. The seawater desalination unit and the carbon dioxide absorption unit are thus matched in terms of performance to the methanol synthesis unit, which has a output capacity of at least 300000 tons, in particular of at least 450000 tons of regeneratively produced methanol per year. The power required to supply the above process units, including the electrolysis unit, is provided by the photovoltaic unit, which is adapted to absorb at least 1500 kWh/m²a, in particular at least 2000 kWh/m²a, in particular at least 2300 kWh/m²a, in particular at least 2500 kWh/m²a, in particular at least 2700 kWh/m²a of solar energy. The unit kWh/m²a means kilowatt hours per square meter and year.

The plant according to the invention forms the basic unit for a larger plant complex with several plants designed in accordance with the plant according to the invention. This allows methanol production to be scaled up in larger quantities so that, with a corresponding number of plants, a large energy requirement, in particular the entire energy requirement of the world's population, can be covered.

Preferred embodiments of the invention are given in the subclaims.

In a preferred embodiment, the photovoltaic unit comprises an effective photovoltaic module area of at least 5 km², in particular at least 7 km², in particular at least 10 km². The plant is thus suitable for regions with large desert or steppe areas, which on the one hand have high and long solar irradiation and on the other hand have large areas available that cannot be used for other purposes.

A particularly effective use of the photovoltaic unit is achieved if the photovoltaic unit comprises photovoltaic modules that comprise solar cells exposed and irradiable on both sides and are arranged at an incline so as to be irradiable directly by sunlight from above and indirectly by sunlight reflected from the ground from below. If the ground is made of light-colored sand or light-colored stones, a particularly good reflection of sunlight is achieved.

Preferably, the carbon dioxide absorption unit comprises at least one chimney and at least one flow channel extending transversely to the chimney and connected to the chimney at a region arranged at the bottom in the installation position. The chimney preferably comprises the air outlet and the flow channel comprises the air inlet. Further preferably, the absorber unit is arranged in the direction of flow between the flow channel and the chimney. The flow channel is preferably elongated and forms an area for supplying ambient air to the absorber unit. The chimney is arranged downstream of the absorber unit and discharges the purified ambient air from the absorber unit into the outside atmosphere.

The chimney can be arranged essentially perpendicular to the flow channel. The air outlet and the air inlet preferably have a height offset from each other. In other words, the air inlet and the air outlet are preferably vertically offset. The absorber unit preferably has ambient air flowing through it. Here, it is advantageous that natural ventilation is realized by the design of the carbon dioxide absorption unit with the chimney and the flow channel, so that no electrically operated fan is required for air acceleration.

Nevertheless, it is possible that in a further embodiment a fan, in particular a blower, is provided to supply ambient air to be cleaned to the absorber unit. This may be necessary, for example, during start-up of the carbon dioxide absorption unit in order to generate the natural chimney draft in the initial phase of operation.

The at least one chimney may has a diameter between 20 meters and 30 meters and a height between 50 meters and 200 meters. The diameter of the chimney refers to the size of the air outlet. It is possible that the chimney has a larger diameter in the flow channel connection area than in the air outlet area. Preferably, the chimney has a diameter of 25 meters and a height of 100 meters. Such dimensions of the chimney enable optimized natural ventilation.

For example, with a diameter of 25 meters and a height of 100 meters of the chimney, and with a first temperature of the ambient air outside the absorption unit of 40° C. and a second temperature of the ambient air inside the absorption unit, especially in the flow channel and/or in the chimney, an air ventilation or an air flow rate with a number of forty chimneys of at least 1800 megatons per year is achieved.

For solar radiation absorption, the flow channel preferably has a surface arranged at the top in the installation position, in particular a surface that is dark-colored at least in sections, in order to heat the ambient air located in the flow channel by radiant heat. The flow channel is preferably arranged directly below the surface arranged at the top. The surface disposed at the top in the installed position may be substantially black. The top arranged surface may be part of at least one sheet. Alternatively, it is possible that the top arranged surface is part of at least one plate. In this case, the natural ventilation for air movement between the flow channel and the chimney is further improved.

In a further embodiment, the surface arranged at the top is dark-colored at least in sections and light-colored at least in sections. This enables absorption and reflection of sunlight.

In a preferred embodiment, the surface arranged at the top is part of a planar plant area, on the long side of which several chimneys, in particular forty chimneys, are arranged in a row, wherein a flow channel runs below the surface arranged at the top towards one of the chimneys in each case. The flow channels may each be separated from one another by a partition wall. The flow channels preferably run parallel and are part of the planar plant area. As a result, the carbon dioxide absorption unit comprises a space-saving and unified structure.

The two-dimensional plant area can be rectangular in plan view. It is also possible that the planar plant area is circular in plan view or has another shape that is adapted to the landscape conditions. The planar plant area is preferably directly adjacent to the other units of the plant in order to keep the lines short.

In one embodiment, the planar plant area is formed by the photovoltaic unit, specifically the photovoltaic modules of the photovoltaic unit, which is arranged on the surface arranged above and is connected to the electrolysis unit and the carbon dioxide absorption unit for power supply. The photovoltaic unit may be formed as a photovoltaic array on the top arranged surface. Preferably, the flow channel is provided through the photovoltaic unit, in particular arranged below the photovoltaic modules. Several flow channels may be provided, the number of which corresponds to several rows of photovoltaic modules. Thus, the number of rows of photovoltaic modules is greater than the number of flow channels. Due to the photovoltaic unit, the plant can be operated self-sufficiently in terms of energy.

Preferably, the seawater desalination unit is adapted to extract a quantity of desalinated water of 1.13 kg from a quantity of seawater of at least 1.5 kg, in particular 2.0 kg.

In a further preferred embodiment, the electrolysis unit is adapted to separate from a water quantity of at least 1.5 kg, in particular of at least 1.7 kg, an oxygen partial quantity of at least 1.2 kg, in particular of at least 1.5 kg, and/or a hydrogen partial quantity of at least 0.1 kg, in particular of at least 0.15 kg. Preferably, the electrolysis unit is adapted to separate from a water quantity of 1.7 kg, an oxygen partial quantity of at least 1.5 kg and a hydrogen partial quantity of at least 0.19 kg. Advantageously, the electrolysis unit is designed to be highly efficient and to produce very large amounts of oxygen and hydrogen.

In a further preferred embodiment, the carbon dioxide absorption unit is adapted to extract from an ambient air quantity of at least 3300 kg a carbon dioxide quantity of at least 1.1 kg to 2 kg, in particular at least 1.4 kg. This enables the significant reduction of the CO₂ concentration in the air.

Preferably, the methanol synthesis unit is adapted to produce a methanol amount (M_{CH3OH}) of 1 kg from a hydrogen amount (M_{H2}) of at least 0.1 kg, in particular at least 0.15 kg, and a carbon dioxide amount (M_{CO2}) of at least 1.1 kg, in particular 1.4 kg.

The methanol synthesis unit and the carbon dioxide absorption unit may be connected to transfer the heat generated during methanol synthesis, which is used as process heat.

Preferably, the electrolysis unit and/or the carbon dioxide absorption unit and/or the methanol synthesis unit each comprise at least one mounting area which can be connected or is connected to a foundation, in particular of a building and/or structure. Through the mounting areas, the electrolysis unit and/or the carbon dioxide absorption unit and/or the methanol synthesis unit are preferably firmly connected to the foundation. Alternatively, the respective unit may be connected to a separate foundation in each case.

When the plant is designed as a large-scale power plant, the respective units are designed on a large scale. The units may each be arranged in a separate operating building. The units may be arranged in separate operating buildings that are directly or indirectly adjacent to each other. Alternatively, the units may each be arranged together in an operations building. A combination of separate arrangement and joint arrangement of the respective units is possible.

The plant may comprise its own infrastructure. For example, the plant may include at least one access road. Further, the plant may comprise a plurality of structures. For example, these may be industrial operating buildings. Additionally, the plant may comprise a port for ships.

A preferred embodiment of the global plant complex according to the invention with several plants according to the invention comprises at least 1800 plants, in particular at least 5000 plants, in particular at least 10000 plants, in particular at least 15000 plants, in particular at least 20000 plants, in particular at least 25000 plants, in particular at least 30000 plants, in particular at least 37000 plants.

With a size of the plant complex of 37000 plants, an area for the photovoltaic unit of approx. 447000 km² is required, if each plant comprises approx. 12 km² of photovoltaic area. The area of 447000 km² corresponds approximately to the area of the Rub′ Al Khali desert in the south of the Kingdom of Saudi Arabia. This area cannot be used for agriculture or other purposes and could be covered with 37000 plants. This would cover the entire world demand for synthetic fuel in the form of methanol, assuming that the world energy demand without already existing renewable energies is about 95.9 PWh (2017) and the annual solar radiation in the Kingdom of Saudi Arabia is about 2400 kWh/mea with an efficiency of about 25% of the photovoltaic unit.

In view of the effect of the invention, which is to enable the world's energy needs to be met in a climate-neutral manner without restricting the existing standard of living and current mobility needs, the land requirement illustrated above is manageable, especially since no other land use is apparent.

The plant complex is not limited to a single geographical region, but can be spread over several regions or continents of the world. Other suitable regions with sufficient solar radiation are, for example, Namibia or Australia.

For the advantages of the method according to the invention, reference is made to the explanations in connection with the plant according to the invention.

Within the scope of the invention, a system for forming a global carbon dioxide cycle with regeneratively produced methanol as energy carrier comprising at least one plant according to the invention is further disclosed and claimed. The system comprises a transport system connected or connectable to the methanol synthesis unit of the plant according to the invention and adapted to transport the methanol regeneratively produced by the methanol synthesis unit from the methanol synthesis unit to at least one output device. The transport system may be stationary or mobile and may include, for example, pumps and pipelines or transport by tankers. Transport systems known per se, for example for the transport of crude oil, can be used. The output device can be a storage tank in a port or at a pumping station.

The system according to the invention comprises a distribution system adapted to distribute the regeneratively produced methanol from the output device to end users for combustion of the regeneratively produced methanol. The distribution system is, for example, in the form of a logistics network in which the methanol is transported to the end users in tankers. Further distribution stations in the form of filling stations or other distribution stations can be provided here. During combustion of the regeneratively produced methanol, carbon dioxide is produced which is released into the atmosphere. According to the invention, the desired carbon dioxide cycle is closed in that the carbon dioxide absorption unit provided in the plant or system according to the invention directly or indirectly separates the carbon dioxide that has entered the atmosphere from it again and uses it for the methanol production method. The methanol thus produced, and thus the carbon dioxide used for its production, is returned to the carbon dioxide cycle.

The fact that the end users and the plants are spatially far apart from each other is irrelevant, since what is at stake is the total balance of carbon dioxide in the atmosphere, which remains constant by forming the closed carbon dioxide cycle. It is even possible to lower the carbon dioxide concentration in the atmosphere if the carbon dioxide absorption unit removes excess carbon dioxide from the atmosphere that is not returned to the cycle by methanol as an energy carrier. The excess carbon dioxide is then supplied elsewhere, for example through storage in deep rock strata, which is already practiced on Iceland.

Potential end users include vehicles, especially hybrid vehicles, aircraft, ships the chemical industry or other industrial sectors in which methanol is used, and cogeneration plants.

Regarding the method according to the invention for forming a global carbon dioxide cycle with regeneratively produced methanol as energy carrier, reference is made to the advantages and effects explained in connection with the system above.

The invention is explained in more detail by means of embodiment examples with reference to the attached schematic drawings with further details.

In these show

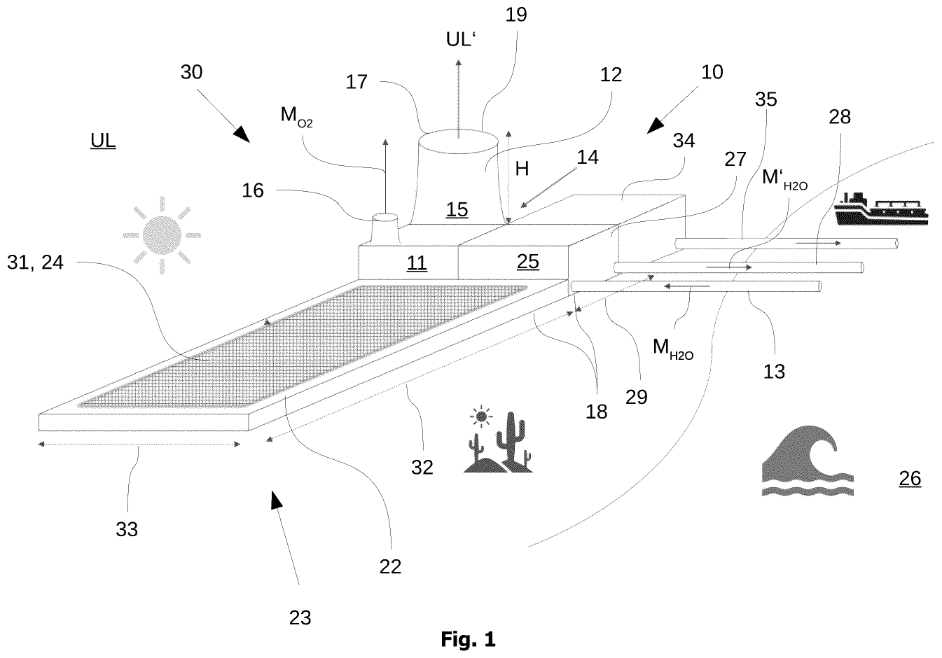

FIG. 1 perspective view of a plant for the production of a globally usable energy carrier according to a preferred embodiment of the invention;

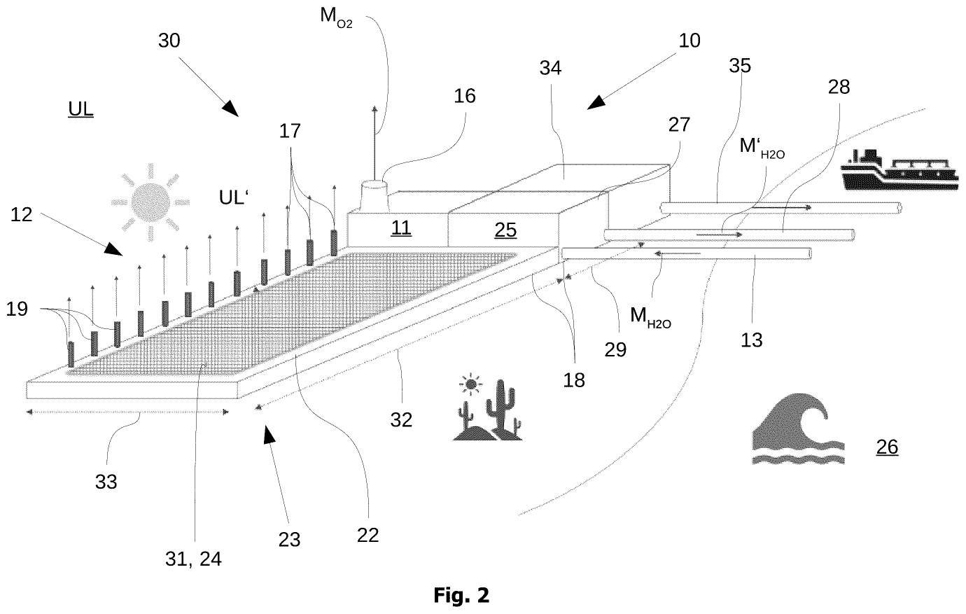

FIG. 2 perspective view of a plant for the production of a globally usable energy carrier according to a further preferred embodiment according to the invention;

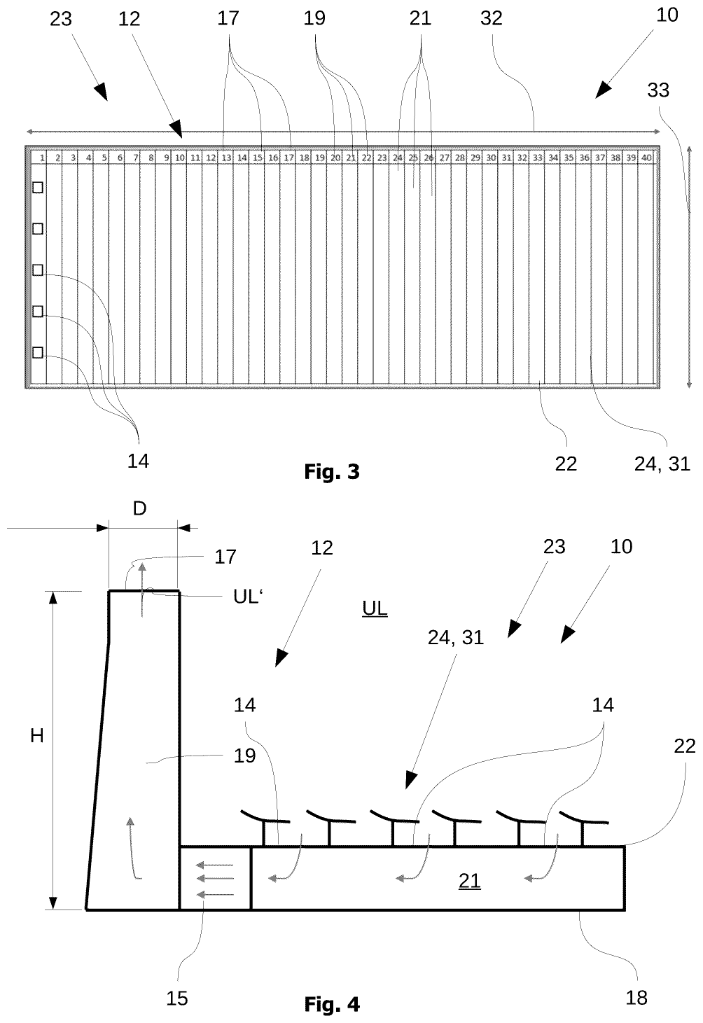

FIG. 3 top view of a planar plant area of the system according to FIG. 2;

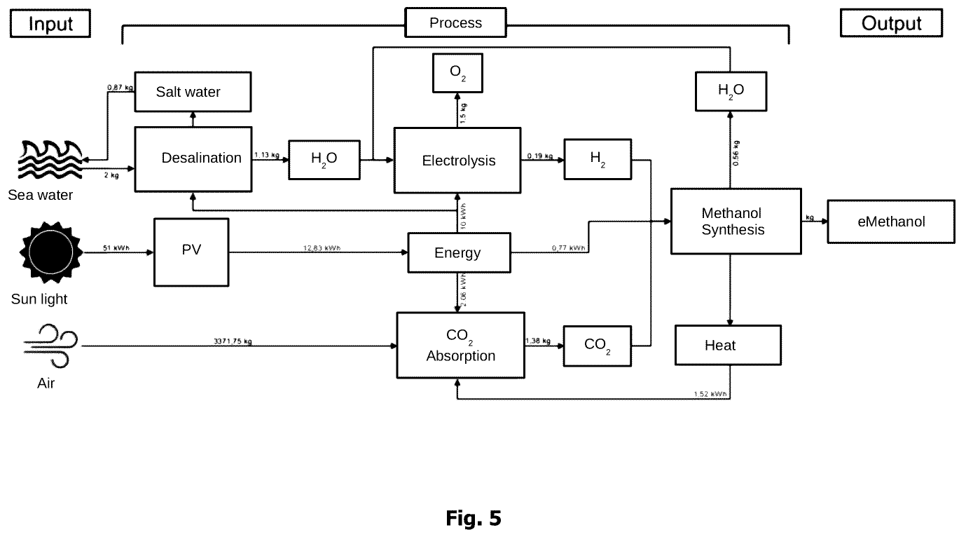

FIG. 4 schematic cross-section through the flat system area of the system according to FIG. 3 and

FIG. 5 flow diagram of the method for the production of a globally usable energy carrier with the plant according to FIG. 1 or the plant according to FIG. 2.

In the following, the same reference numerals are used for identical and identically acting parts.

FIG. 1 shows an embodiment of a plant 10 designed to produce a globally usable energy carrier in the form of methanol. The plant 10 comprises the following components, namely an electrolysis unit 11, a carbon dioxide absorption unit 12, a seawater desalination unit 27, and a methanol synthesis unit 34. For supplying power to the aforementioned units, a power generation unit 31 in the form of a photovoltaic unit 24 is provided, which is connected to the respective units 11, 12, 27, 34 for supplying power.

Instead of the seawater desalination unit 27, another water supply unit can be provided which supplies the electrolysis unit 11 with salt-free water obtained, for example, from a river or lake. For the required water quantities, the seawater desalination unit 27 provided in the example shown in FIG. 1 is particularly advantageous, since unlimited quantities of water can be taken from the adjacent sea shown in FIG. 1 as a water reservoir.

As can be seen from FIG. 1, the above-mentioned plant components are arranged on a contiguous plant site so that the exchange of material flows between the various units and the power supply take place with the lowest possible losses. The shape of the plant is not limited to that shown in FIG. 1.

The electrolysis unit 11 is connected to the seawater desalination unit 27 by at least one pipeline (not shown) for the supply of water, in particular desalinated water. The desalinated water is supplied to the electrolysis unit 11 through the pipeline. The methanol synthesis unit 34 is connected, on the one hand, to the electrolysis unit 11 by at least one pipeline and, on the other hand, to the carbon dioxide absorption unit 12 by at least one further pipeline. Through the two pipelines, the hydrogen produced in the electrolysis unit 11 and the carbon dioxide separated in the carbon dioxide absorption unit 12 are fed to the methanol synthesis unit 34. Methanol is produced therefrom in the methanol synthesis unit 34. The seawater desalination unit is designed to receive and desalinate at least 900000 tons of seawater per year. The carbon dioxide absorption unit is designed to have an extraction capacity of at least 400000 tons of carbon dioxide per year, and in particular at least 600000 tons of carbon dioxide per year, extracted from ambient air. The methanol synthesis unit 34 is adapted to produce at least 300000 tons, in particular 450000 tons of regeneratively produced methanol per year.

The photovoltaic unit 24 comprises a power of approx. 1.5 GW and can absorb at least 1500 kWh/mea, depending on the solar radiation. For the location in the Middle East selected in FIG. 1, the photovoltaic unit 24 is adapted to absorb at least 2500 kWh/m²a. The electrolysis unit 11 is designed to separate a quantity of water M_{H2O} by electrolysis into a partial quantity of oxygen M_{O2} and a partial quantity of hydrogen. The electrolysis unit 11 thus forms a unit for water electrolysis. The electrolysis unit 11 is connected to a water supply line 13 for receiving the water quantity M_{H2O}. As can be seen in FIG. 1, a pump unit 25 is arranged between the electrolysis unit 11 and the water supply line 13. The pump unit 25 comprises at least one pump for conveying water from a water reservoir 26. The water reservoir 26 may be a sea with sea water. Alternatively, the water reservoir 26 may be a lake with fresh water. It is also possible that the water supply line 13 is connected to a river to draw fresh water for water electrolysis. In the plant 10 shown in FIG. 1, the water supply line 13 is connected to a sea for taking sea water. The plant 10 is located near the coast to keep the distance to be covered to the water supply, in particular the water supply line 13 short.

The pump unit 25 is designed to pump seawater from the sea and make it available to further plant parts or units for further processing. In order to prepare the seawater for the electrolysis process by the electrolysis unit 11, the plant 10 comprises a seawater desalination unit 27. The seawater desalination unit 27 is connected to the pump unit 25 by at least one pipeline. The seawater desalination unit 27 is adapted to separate out a certain amount of salt from the pumped seawater M_{H2O}, so that the seawater comprises a reduced salt content after the desalination process by the seawater desalination unit 27. The desalinated seawater amount M_{H2O} corresponds to the water amount M_{H2O}, which is separated into an oxygen partial amount M_{O2} and a hydrogen partial amount by the electrolysis unit 11. The electrolysis unit 11 is connected to the seawater desalination unit 27 by at least one pipeline. In order to convey the desalinated seawater from the seawater desalination unit 27 to the electrolysis unit 11, at least one further pump can be interposed.

As described above, the electrolysis unit 11 is adapted to separate the absorbed water quantity M_{H2O} into a hydrogen partial quantity and an oxygen partial quantity M_{O2}. The hydrogen partial amount is supplied to the methanol synthesis unit 34. The oxygen partial amount M_{O2} is discharged to the environment. The electrolysis unit 11 is adapted to separate an oxygen partial quantity M_{O2} of at least 1.2 kg and a hydrogen quantity of at least 0.15 kg, in particular of 0.19 kg, from an absorbed water quantity M_{H2O} of at least 1.5 kg. For discharging the generated partial oxygen quantity M_{O2}, the electrolysis unit 11 comprises an oxygen outlet 16 which opens into the outside atmosphere. The plant 10 comprises a hydrogen transport device for supplying the hydrogen to the methanol synthesis unit 34, which is not shown.

It is possible for the plant 10 to comprise a hydrogen storage system so that the methanol synthesis unit 34 can be supplied with hydrogen as continuously as possible.

Referring to FIG. 1, the carbon dioxide absorption unit 12 comprises an air inlet 14 for supplying ambient air UL and a downstream absorber unit 15. It is possible that the carbon dioxide absorption unit 12 comprises one or more air inlets 14. The absorber unit 15 is connected to the air inlet 14. The absorber unit 15 is adapted to extract an amount of carbon dioxide from the ambient air UL. The carbon dioxide absorption unit 12 further comprises an air outlet 17 oriented upward in the vertical direction. The air outlet 17 is for discharging the exhaust air UL′ having a carbon dioxide concentration lower than the carbon dioxide concentration of the ambient air UL. The air outlet 17 is part of a chimney 19.

Specifically, the absorber unit 15 is disposed between the air inlet 14 and the air outlet 17. In operation, the ambient air UL flows through the air inlet 14 to the absorber unit 15, which separates, in particular filters, a certain amount of carbon dioxide from the air UL, wherein the filtered exhaust air UL′ flows after the absorber unit 15 through the air outlet 17 into the outside atmosphere. Generally, it is possible that a plurality of air inlets 14, a plurality of absorber units 15, and a plurality of air outlets 17 are provided.

Specifically, FIG. 1 shows a single chimney 19 with a height H of 200 meters, which exemplifies the external structure of the carbon dioxide absorption unit 12. The air outlet 17, as shown in FIG. 1, also discharges into the outside atmosphere, as does the oxygen outlet 16.

The plant 10 further comprises a carbon dioxide transport device, not shown, which is designed to make the carbon dioxide quantity separated from the ambient air UL available to a carbon dioxide storage unit and/or the methanol synthesis unit 34 for further processing. The carbon dioxide storage unit serves to ensure as continuous a supply of carbon dioxide as possible to the methanol synthesis unit 34.

The carbon dioxide absorption unit 12 comprises an extraction capacity of an amount of carbon dioxide per year of at least 400000 tons, in particular 600000 tons. In other words, the carbon dioxide absorption unit 12 is adapted to process an amount of ambient air per year of at least 1500 megatons. Specifically, the carbon dioxide absorption unit 12 is adapted to extract a carbon dioxide amount of at least 1.4 kg from an ambient air amount of at least 3300 kg.

As shown in FIG. 1, the plant 10 comprises a planar plant area 23. The planar plant area 23 is directly connected to the electrolysis unit 11. A power generation unit 31, which is a photovoltaic unit 24, is arranged on the planar plant area 23. The photovoltaic unit 24 is connected to the respective units of the plant 10 for power supply. The photovoltaic unit 24 is adapted in such a way that the entire plant 10 or the entire system 30 can be operated self-sufficiently in terms of energy. This is to be understood as meaning that the electrical power for operating the entire plant 10 is provided exclusively by solar energy by means of the photovoltaic unit 24. In other words, no fossil energy sources are used to operate the plant 10.

The areal plant area 23 has a longitudinal extension 32 of about 5000 meters and a transverse extension 33 of about 2000 meters. In other words, the area of the plant 10 covers an area of 10 square kilometers. The plant area shown in FIG. 1 containing the electrolysis unit 11 may has a partial longitudinal extension 29 of approximately two kilometers. Other partial longitudinal, longitudinal and transverse extents 29, 32, 33 are possible.

The seawater desalination unit 27 described above is connected to a water return line 28, through which a returnable seawater quantity M′_{H2O} with increased salt content is returned to the sea. Specifically, a certain salinity is extracted from the extracted seawater quantity and then returned to the sea with a portion of the extracted seawater quantity as a returnable water quantity M′_{H2O}. This provides a water cycle that is harmless to nature.

The preferred installation site of the plant 10 is near the coast of a sea. Particularly preferably, the plant 10 is set up in a desert. The plant 10 according to FIG. 1 is a large-scale power plant. The plant 10 comprises at least one mounting area 18 connected to a foundation of a building and/or a structure. Generally, it is possible that the electrolysis unit 11 and/or the carbon dioxide absorption unit 12 are arranged in a common building or in separate buildings.

The power supply unit 31 preferably comprises a power storage unit, not shown, adapted to supply power to the plant 10 during nighttime operation.

In contrast to FIG. 1, FIG. 2 shows a plant 10 in which the single carbon dioxide absorption unit 12 is replaced by several carbon dioxide absorption units 12. The respective carbon dioxide absorption unit 12 according to FIG. 2 comprises a chimney 19 and a flow channel 21 extending transversely to the chimney 19. This is clearly visible in FIG. 4, for example. The flow channel 21 is connected to the chimney 19 at a region of the chimney arranged at the bottom in the installation position. An absorber unit 15 is arranged between the flow channel 21 and the chimney 19, which is designed to extract a quantity of carbon dioxide from ambient air UL. The absorber unit 15 is formed by an amine exchanger. Other types of absorber units are possible.

As shown in FIGS. 2, 3, the chimneys 19 are arranged along the longitudinal extension 32 of the planar plant area 23. The planar plant area 23 comprises a surface 22 arranged at the top in the installation position. The surface 22 arranged at the top is dark-colored, at least in sections, in order to absorb solar energy. The flow channels 21 are arranged below the surface 22 arranged at the top in the installation position. A plurality of air inlets 14 are formed in the upper arranged surface 22 for supplying ambient air UL into the flow channels 21. The air inlets 14 form through openings through the surface 22 arranged above. These are shown in FIG. 4 for the sake of better illustration only at the first flow channel 21. Likewise, the number of air inlets 14 is variable.

In operation, ambient air flows through the air inlets 14 into the flow channel 21 and then through the absorber unit 15. After the absorber unit 15, the exhaust air UL′ with reduced carbon dioxide concentration flows into the chimney 19 and through the air outlet 17 into the outside atmosphere. Due to the dark-colored surface 22 located at the top, the ambient air located below the surface 22 in the flow channel 21 heats up during operation. The temperature of the ambient air UL in the flow channel 21 is preferably about 60° C. When the outside temperature of the ambient air UL is about 40° C., natural ventilation is generated by the arrangement of the chimney with the flow channel 21 as well as the dark-colored surface 22. In other words, no fan or blower is necessary for the supply of the ambient air UL into the flow channel 21 as well as for the flow through the absorber unit 15 and the outflow of the purified ambient air UL′ from the chimney 19.

According to FIG. 3, a top view of the planar plant area 23 of the plant 10 according to FIG. 2 is shown. The numbering from 1 to 40 shown along the longitudinal extension 32 represents the number of chimneys 19 arranged along the longitudinal extension 32. The lines running transversely to the longitudinal extension 32 show schematic separations between the individual flow channels 21. The individual flow channels 21 are each assigned to a chimney 19. In each case, an absorber unit 15 is arranged between the flow channel 21 and the chimney 19. The longitudinal extent 32 of the two-dimensional plant area 23 is approximately 5000 meters and the transverse extent 33 of the two-dimensional plant area 23 is approximately 2000 meters. A total of forty chimneys 19 with a total of forty flow channels 21 are provided in the two-dimensional plant area 23. These have a combined discharge capacity of exhaust air UL′ of at least 1800 megatons per year.

To achieve this, the chimneys 19 comprise a diameter D which is 25 meters. The diameter D refers to that area of the chimney 19 in which the air outlet 17 is formed. The air outlet 17 is formed at a free end of the chimney 19. Furthermore, the respective chimney 19 comprises a height H of 100 meters. Thus, an optimal shape for the chimney effect for natural ventilation is formed. Other dimensions of the chimneys 19 are possible.

Furthermore, more or less than forty chimneys 19, each with an associated flow channel 21, may be arranged in the planar plant area 23.

As can be seen in FIG. 4, the planar plant area 23 is provided with a photovoltaic unit 24 on the surface 22 arranged at the top. In other words, a photovoltaic unit 24 is arranged on the top arranged surface 22 of the planar plant area 23. The photovoltaic unit 24 preferably comprises a power of 1.5 gigawatts per year. In the system 30 according to FIG. 2, the carbon dioxide absorption unit 12 and the photovoltaic unit 24 thus spatially form a common unit. The photovoltaic unit 24 forms a power supply unit 31 for energy-autonomous operation of the entire plant 10.

It should be noted that the above-described plants 10 and systems 30 shown in FIGS. 1 and 2 are identical except for the differences described.

The method that can be carried out with the plant 10 according to FIG. 1 or FIG. 2 is explained with reference to the flow chart according to FIG. 4:

To produce a quantity of 1 kg of methanol, a quantity of approximately 2 kg of seawater is supplied to the plant 10 and desalinated in the seawater desalination unit 27. This produces about 1.13 kg of desalinated water. The residual salt water (about 0.87 kg) is returned to the sea through the water return line 28. In the electrolysis unit, the desalinated water and, if necessary, further quantities of water produced in subsequent process steps are split into hydrogen (approx. 0.19 kg) and oxygen (approx. 1.5 kg). The carbon dioxide absorption unit 12 takes in an amount of air of about 3371.75 kg through the air inlet 14 and extracts an amount of carbon dioxide of about 1.38 kg therefrom. Hydrogen and carbon dioxide are fed to the methanol synthesis unit where they are processed to produce 1 kg of methanol. The excess heat generated during the synthesis is fed to the carbon dioxide absorption unit 12. The synthesis further produces water in an amount of about 0.56 kg, which is fed to the electrolysis unit. For these process steps, the photovoltaic system converts approx. 51 kWh of solar energy into approx. 12.83 kWh of usable electricity energy.

The invention offers, as explained by the above embodiments, a technically feasible and economical solution to the acute climate problem, which can be implemented in a reasonable time frame due to the scalability of the described plants. The invention takes into account the geographical opportunities offered by certain regions of the world and is impressive in its simplicity.

### LIST OF REFERENCE SIGNS

10 plant
11 electrolysis unit
12 carbon dioxide absorption unit
13 water supply line
14 air inlet
15 absorber unit
16 oxygen outlet
17 air outlet
18 mounting area
19 chimney
21 flow channel
22 top arranged surface
23 planar plant area
24 photovoltaic unit
25 pump unit
26 water reservoir
27 sea water desalination unit
28 water return line
29 partial longitudinal extension
30 system
31 power generation unit
32 longitudinal extension
33 transverse extension
34 methanol synthesis unit
35 methanol output line
UL environment air with increased carbon dioxide concentration
UL′ exhaust air with reduced carbon dioxide concentration
D diameter
H height
M_{H2O} quantity of water
M′_{H2O} recirculated water quantity
M_{O2} partial oxygen quantity

1-20. (canceled)

21. A production plant for a globally usable energy carrier comprising:
a photovoltaic unit for converting solar energy into electricity and having a capacity of at least 1.0 gigawatt;
a water supply unit having a seawater desalination unit for production of desalinated water having an intake capacity of at least 900,000 tons of seawater per year;
an electrolysis unit for the production of hydrogen connected by at least one pipeline to the desalinated water of the water supply unit;
a carbon dioxide absorption unit for absorbing carbon dioxide from ambient air and having an extraction capacity of at least 400,000 tons of carbon dioxide per year; and
a methanol synthesis unit for producing methanol connected by a first pipeline to the electrolysis unit for supplying hydrogen and connected by a second pipeline to the carbon dioxide absorption unit for supplying carbon dioxide, wherein the methanol synthesis unit and the carbon dioxide absorption unit are connected for transferring heat generated during methanol synthesis, wherein the water supply unit, the electrolysis unit, the carbon dioxide absorption unit and the methanol synthesis unit each are connected to the photovoltaic unit for supplying electrical power and are further configured and arranged in a contiguous plant area aboard the energy carrier, wherein the photovoltaic unit is configured and adapted to capture at least 1500 kWh/m²a of solar energy, and wherein the methanol synthesis unit has an output capacity of at least 300,000 tons of regeneratively produced methanol per year.

22. The production plant according to claim 21, wherein the photovoltaic unit has an effective photovoltaic module area for capturing solar radiation of at least 5 km².

23. The production plant according to claim 22, wherein the photovoltaic unit further comprises two-sided photovoltaic modules configured and arranged on an incline so as to be irradiated directly by sunlight from above and irradiated indirectly by sunlight reflected from below.

24. The production plant according to claim 23 wherein the carbon dioxide absorption unit further comprises at least one chimney and at least one flow channel which extends transversely to the chimney and is connected to the chimney at a region arranged at a bottom in an installation position, wherein the chimney includes an air outlet, wherein the flow channel includes an air inlet, and wherein the absorber device is arranged between the air inlet and the air outlet in a flow direction.

25. The production plant according to claim 24, wherein the at least one chimney has a diameter between 20 meters and 30 meters, and has a height between 50 and 200 meters.

26. The production plant according to claim 24, wherein the flow channel passes under the photovoltaic modules.

27. The production plant according to claim 26, further comprising a plurality of flow channels having a quantity corresponding to a plurality of rows of the photovoltaic modules included in the photovoltaic unit.

28. The production plant according to claim 21, wherein the seawater desalination unit is configured and adapted to extract a quantity of desalinated water of 1.13 kg from a quantity of seawater of at least 1.5 kg.

29. The production plant according to claim 21, wherein the electrolysis nit is configured and adapted to separate from a quantity of water of at least 1.5 kg, a partial quantity of oxygen of at least 1.2 kg and a partial quantity of hydrogen of at least 0.1 kg.

30. The production plant according to claim 21, wherein the carbon dioxide absorption unit is configured and adapted to extract from an ambient air quantity of at least 3300 kg a carbon dioxide quantity of at least 1.1 kg.

31. The production plant according to claim 21, wherein the methanol synthesis unit is configured and adapted to produce a quantity of methanol of 1 kg from a quantity of hydrogen of at least 0.1 kg and a quantity of carbon dioxide of at least 1.1 kg.

32. The production plant according to claim 21, wherein the production to plant is a global plant complex comprising at least 1800 independently producing plants.

33. A method for production in a production plant of a globally usable energy carrier comprising:
converting solar energy into electricity using a photovoltaic unit at a peak is power of at least 1.0 gigawatt absorbing at least 1500 kWh/m e a of solar energy;
producing desalinated water from at least 900,000 tons of seawater per year using a desalination unit supplied with electricity by the photovoltaic unit;
producing hydrogen from the desalinated water using an electrolysis unit supplied with electricity by the photovoltaic unit and supplied with desalinated water via a first pipeline from the desalination unit;
absorbing at least 400,000 tons of carbon dioxide from ambient air via a carbon dioxide absorption unit supplied with electricity by the photovoltaic unit;
synthesizing at least 300,000 tons per year of methanol regeneratively using a methanol synthesis unit supplied with hydrogen through a second pipeline from the electrolysis unit and supplied with carbon dioxide through a third pipeline from the carbon dioxide absorption unit, and supplied with electricity from the photovoltaic unit, wherein the methanol synthesis unit and the carbon dioxide absorption unit are connected for transferring heat generated during methanol synthesis.

34. The method of production for the production plant according to claim 33, further comprising using the synthesized methanol as fuel for mobility applications and cogeneration plants.

35. A system for the formation of a global carbon dioxide cycle with regeneratively produced methanol as an energy carrier comprising:
a photovoltaic unit for converting solar energy into electricity and having a capacity of at least 1.0 gigawatt;
a water supply unit having a seawater desalination unit for production of desalinated water having an intake capacity of at least 900,000 tons of seawater per year;
an electrolysis unit for the production of hydrogen connected by at least one pipeline to the desalinated water of the water supply unit;
a carbon dioxide absorption unit for absorbing carbon dioxide from ambient air and having an extraction capacity of at least 400,000 tons of carbon dioxide per year;
a methanol synthesis unit for regeneratively producing methanol connected by a first pipeline to the electrolysis unit for supplying hydrogen and connected by a second pipeline to the carbon dioxide absorption unit for supplying carbon dioxide, wherein the methanol synthesis unit and the carbon dioxide absorption unit are connected for transferring heat generated during methanol synthesis, wherein the water supply unit, the electrolysis unit, the carbon dioxide absorption unit and the methanol synthesis unit each are connected to the photovoltaic unit for supplying electrical power and are further configured and arranged in a contiguous plant area aboard the energy carrier, wherein the photovoltaic unit is configured and adapted to capture at least 1500 kWh/m²a of solar energy, and wherein the methanol synthesis unit has an output capacity of at least 300,000 tons of regeneratively produced methanol per year; and
a transport system connectable to the methanol synthesis unit and adapted to transport the regeneratively produced methanol from the methanol synthesis unit to at least one of (i) an output device or (ii) a distribution system configured and adapted to distribute the transported methanol from the output device to end users for combustion, wherein the carbon dioxide produced and released into atmosphere during combustion of the transported methanol is removable from the atmosphere directly or indirectly by the carbon dioxide absorption unit.

36. The system according to claim 35, wherein the end users include one or more of vehicles, aircraft, ships, chemical industry plants or cogeneration plants.

37. A method for formation of a global carbon dioxide cycle with regeneratively produced methanol as an energy carrier comprising:
converting solar energy into electricity using a photovoltaic unit at a peak power of at least 1.0 gigawatt absorbing at least 1500 kWh/m²a of solar energy;
producing desalinated water from at least 900,000 tons of seawater per year using a desalination unit supplied with electricity by the photovoltaic unit;
producing hydrogen from the desalinated water using an electrolysis unit supplied with electricity by the photovoltaic unit and supplied with desalinated water via a first pipeline from the desalination unit;
absorbing at least 400,000 tons of carbon dioxide from ambient air via a carbon dioxide absorption unit supplied with electricity by the photovoltaic unit;
synthesizing at least 300,000 tons per year of methanol regeneratively using a methanol synthesis unit supplied with hydrogen through a second pipeline from the electrolysis unit and supplied with the carbon dioxide through a third pipeline from the carbon dioxide absorption unit, and supplied with electricity from the photovoltaic unit; and transporting the synthesized methanol via a transport system connectable to the methanol synthesis unit and adapted to transport the synthesized methanol from the methanol synthesis unit to at least one of (i) an output device or (ii) a distribution system configured and adapted to distribute the transported synthesized methanol from the output device to end users for combustion, wherein the carbon dioxide produced and released into atmosphere during combustion of the transported synthesized methanol is removable from the atmosphere directly or indirectly by the carbon dioxide absorption unit.
